(19) United States
(12) Patent Application Publication
(10) Pub. No.: US 2026/0028378 A1
(43) Pub. Date: Jan. 29, 2026

(54) CHEMICALLY MODIFIED ENGINEERED SPIDER SILK PROTEINS

(71) Applicant: LATVIAN INSTITUTE OF ORGANIC SYNTHESIS, Riga (LV)

(72) Inventors: Kristaps Jaudzems, Riga (LV); Viktors Romanuks, Riga (LV); Jekabs Fridmanis, Riga (LV); Anna Lina Bula, Ikskile (LV); Gints Smits, Carnikava (LV)

(21) Appl. No.: 18/867,525

(22) PCT Filed: May 26, 2022
(86) PCT No.: PCT/IB2022/054946

§ 371 Date: Nov. 20, 2024

(51) Int. Cl. C07K 14/435; A61K 38/00; C12N 5/00

(52) U.S. Cl. CPC C07K 14/43518; A61K 38/00; C12N 5/0068; C07K 2319/00; C12N 2533/50

(57) ABSTRACT

The present invention relates to recombinant spidroin bioconjugates (chemically modified engineered spider silk proteins). More particularly, the invention relates to recombinant spidroin bioconjugates with poly-ethyleneglycol polymers and their use in regenerative medicine.

### FIELD OF INVENTION

The present invention relates to recombinant spidroin bioconjugates (chemically modified engineered spider silk proteins). More particularly, the invention relates to recombinant spidroin bioconjugates with poly-ethyleneglycol polymers and their use in regenerative medicine.

### BACKGROUND OF INVENTION

Spider silk is made of proteins (spidroins) that generally consist of three structural subunits, the non-repetitive N- and C-terminal domains (NT and CT) and a long central region with highly repetitive sequences (REP). The NT and CT are implicated in the silk formation process, while the central region determines the mechanical properties of the silk fibers.

Recombinant spidroins can be produced using heterologous expression in bacterial, yeast and other expression systems. The recombinant spidroins usually differ from the natural spidroins in that they contain a lower number of repeat sequences (Rep) in their central repetitive region and often lack one or both terminal domains.

The structural transition of the spidroins from soluble dope to solid fibers is mediated by changes in pH and ion composition of the aqueous environment as well as shear forces. Studies of the terminal domains have shown that upon lowering of pH to about 5.5, the NT dimerizes and becomes stabilized, thereby firmly interconnecting the spidroins, whereas CT unfolds and forms amyloid-like fibrils that may function as nucleation seeds facilitating the conversion of the repetitive region to β-sheet structures (Andersson et al. PLOS Biol. 2014, 12, e1001921).

Recombinant spidroins lacking one or both terminal domains are not capable of reproducing the molecular mechanisms of native silk spinning, in particular, the structural transition of the terminal domains. Their conversion to β-sheet structures therefore requires non-native methods, for example, coagulation. Obtaining artificial spider silk in a biomimetic way requires that the recombinant spidroins contain both terminal domains and that conditions in the spider silk gland are precisely reproduced to ensure the recombinant spidroin assembly.

Recombinant spidroins containing both of the terminal domains and being able to polymerize in a biomimetic way to form spider silk-like fibers and methods for producing such fibers have been described in EP 3263593.

The present invention is aimed at circumventing the requirement for both terminal domains in the genetic construct of recombinant spidroins to still produce spider silk-like fibers in a biomimetic way.

### SUMMARY OF THE INVENTION

In a first aspect, the invention relates to a recombinant spidroin, comprising from 244 to 620 amino acid residues and defined by the formula NT-(Rep)ₓ-C comprising three features:
a. N-terminal domain NT, which consists of fragment of 130 to 156 amino acid residues derived from N-terminal domain of a spidroin;
b. a repetitive domain (Rep)ₓ, which consists of 87 to 463 amino acid residues derived from repetitive sequences in a spidroin; where the x is the number of repetitive sequences; (Rep)ₓ-domain preferably consists of 174-463 amino acid residues;
c. a domain C is a Cys(Z) residue, where Z is optional, if present, it is selected from: Ala, Arg, Asn, Asp, Cys, Glu, Gln, Gly, His, Ile, Leu, Lys, Met, Phe, Pro, Ser, The, Trp, Tyr, Val.
   Cysteine is positioned at the C-terminus or one residue before the C-terminal residue, which can be used for bioconjugation to a polymer.

In another aspect, the invention features a method of bioconjugation of the recombinant spidroins to a poly-ethyleneglycol polymer comprising the following steps:
a. Disulfide bond reduction to obtain free thiol groups with tris(2-carboxyethyl)phosphine (TCEP); Reaction temperature is in range from 2 to 10° C. and pH is in the range from 6.0 to 9.0. Preferably the reaction temperature is in range from 3 to 5° C. and pH is in range from 7.2 to 7.5.
b. Quenching with 1,2-bis(2-azidoethoxy) ethane
c. Coupling with 2- to 8-arm polyethylene glycol maleimide (PEG-Mal)
d. Forming recombinant spidroin bioconjugates.

Obtained assembled polymers are analysed by sodium dodecyl sulfate-polyacrylamide gel electrophoresis (SDS-PAGE).

Obtained bioconjugated recombinant spidroin comprises from 244 to 623 amino acid residues and is defined by the formula (NT-(Rep)ₓ-C)_{y}-PEG-Mal wherein:
an N-terminal domain NT consisting of 130 to 156 amino acid residues derived from the N-terminal domain of a spidroin;
a repetitive domain (Rep)ₓ consists from 87 to 463 amino acid residues derived from the repetitive sequences in a spidroin, wherein x is a number of repetitive sequences from 1 to 8;
a domain C is Cys (Z) residue, where Z is optional, if present, selected from the group Ala, Arg, Asn, Asp, Cys, Glu, Gln, Gly, His, Ile, Leu, Lys, Met, Phe, Pro, Ser, The, Trp, Tyr, Val;
y is a number of bioconjugated NT-(Rep)ₓ-C proteins from 1 to 8;
PEG-Mal is 2- to 8-arm polyethylene glycol maleimide;
and optical isomers thereof.

In one embodiment a bioconjugated recombinant spidroin is a compound with a general formula (NT-(Rep)ₓ-C)_{y}-PEG-Mal, and has the following specific structures I-III:

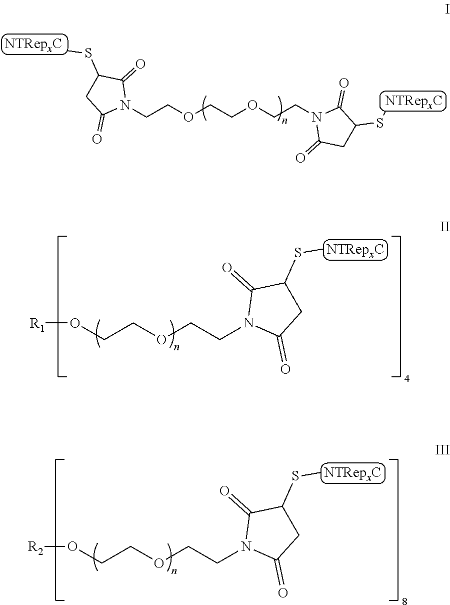

wherein:
R₁=pentaerythritol core;
R₂=tripentaerythritol core;
n is an integer ranging from 50 to 400, corresponding to an average molecular weight between 5000 Da and 10000 Da;
x is a number of repetitive sequences from 1 to 8;
domain C is Cys(Z) residue where Z is optional, if present, is selected from the group Ala, Arg, Asn, Asp, Cys, Glu, Gln, Gly, His, Ile, Leu, Lys, Met, Phe, Pro, Ser, The, Trp, Tyr, Val;
y is a number of bioconjugated NT-(Rep)ₓ-C proteins from 1 to 8;
and optical isomers thereof.

In another aspect, the invention features the use of recombinant spidroin bioconjugates for producing a fiber in a biomimetic way, which can be used in regenerative medicine, or as a cell culture scaffold.

In one embodiment, the invention features a fiber obtained from chemically modified recombinant spidroin derivatives described herein.

In another aspect, the invention features compounds obtainable by a method of synthesis as described herein, or a method comprising a method of synthesis as described herein.

In another aspect, the invention features compounds obtained by a method of synthesis as described herein, or a method comprising a method of synthesis as described herein.

In another aspect, the invention features novel intermediates, as described herein, which are suitable for use in the methods of synthesis described herein.

As will be appreciated by person skilled in the art, features and preferred embodiments of one aspect of the invention will also pertain to other aspects of the invention.

### DESCRIPTION OF DRAWING

FIG. 1 shows compounds prepared by the method described in Synthesis of products 1.1-1.15, general method.

### DESCRIPTION OF INVENTION

Recombinant spidroins containing NT domain have been identified as promising biopolymers that can be expressed in high yield in bacterial cultures, that have high solubility and allow spinning of artificial spider silk fibres without use of organic solvents (Andersson et al. Nat. Chem. Biol. 2017, 13, 262-264).

Bioconjugation of recombinant spidroins having the general formula NT-(Rep)ₓ-C(NT-(Rep)ₓ-Cys or NT-(Rep)ₓ-CysAla) with 2- to 8-arm polyethylene glycol maleimide (PEG-Mal) was used to produce recombinant spidroin bioconjugates (NT-(Rep)ₓ-C)_{y}-PEG-Mal. The formed products are dimers to octamers linked depending on the number of arms for PEG-Mal, which can be further polymerized by NT-mediated dimerization at low pH. Thus, the method enables crosslinking of recombinant spidroins to a variable extent by varying the bioconjugation reaction conditions.

According to this invention, the results from fiber spinning experiments demonstrate that artificial spider silk fibers can be produced by extruding chemically modified recombinant spidroin into a low pH solution.

### Combinations

Each and every compatible combination of the embodiments described above is explicitly disclosed herein, as if each and every combination was individually and explicitly recited.

### Examples of Specific Embodiments

The following examples further illustrate the invention, but should not be construed to limit the scope of the invention in any way.

The following recombinant spidroins bioconjugates with general formulas (NT-(Rep)ₓ-C)_{y}-PEG-Mal were prepared as examples of the current invention:

| | Cmpd. | |
|---|---|---|
| ID | No | Structure |
| F2M-2arm SEQ ID: 1 | 1.1 | 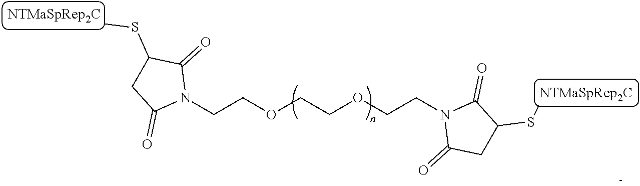 |
| F2M-4arm SEQ ID: 1 | 1.2 | 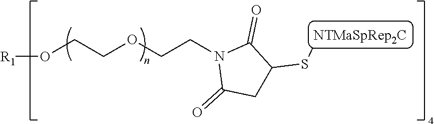 |
| F2M-8arm SEQ ID: 1 | 1.3 | 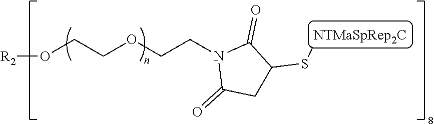 |
| F4M-2arm SEQ ID: 2 | 1.4 | 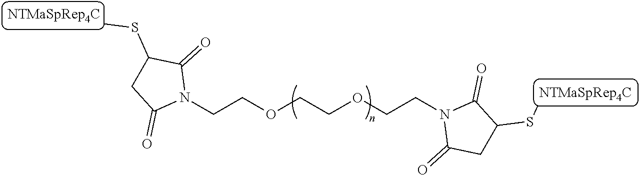 |
| F4M-4arm SEQ ID: 2 | 1.5 | 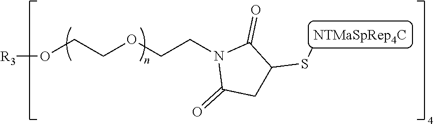 |
| F6M-2arm SEQ ID: 3 | 1.6 | 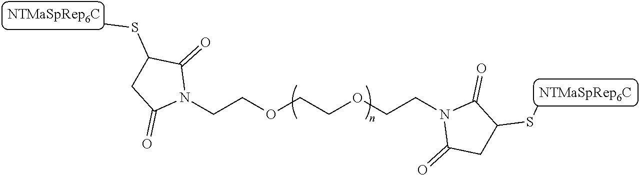 |
| F6M-4arm SEQ ID: 3 | 1.7 | 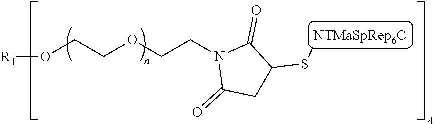 |
| F2M*- 4arm SEQ ID: 4 | 1.8 | 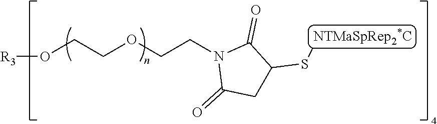 |
| F1F-2arm SEQ ID: 5 | 1.9 | 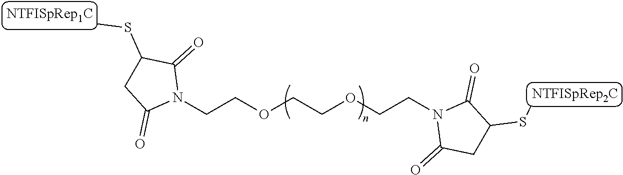 |
| F1F-4arm SEQ ID: 5 | 1.10 | 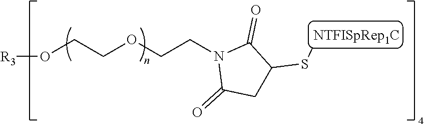 |
| F1F-8arm SEQ ID: 5 | 1.11 | 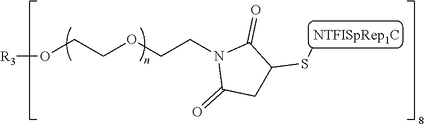 |
| F1F*-4arm SEQ ID: 6 | 1.12 | 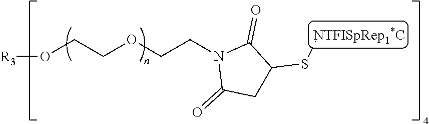 |
| F1T-2arm SEQ ID: 7 | 1.13 | 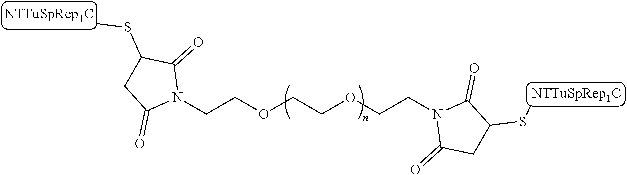 |
| F1T*-2arm SEQ ID: 8 | 1.14 | 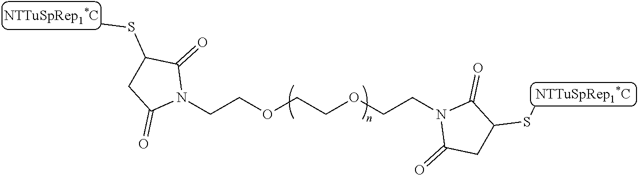 |
| F1T-4arm SEQ ID: 7 | 1.15 | 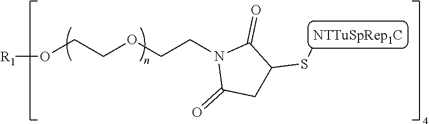 |

### Protein Expression and Purification

The constructs F2M, F4M, F6M, FIF, F2M*, FIT are composed of His-tag (MGKHHHHHHPMSDYDIPTT) and Tobacco etch virus (TEV) protease cleavage site (ENLYFQG) followed by N-terminal from domain N. clavipes F1Sp (IANSPFSNPNTAEAFARSFVSNIVSSGEFGAQGAEDFEDIIQSLIQAQSMGKGRHDTKAK AKAMQVALASSIAELVIAESSGGDVQRKTNVISNALRNALMSTTGSPNEEFVHEVQDLIQ MLSQEQINEV), a repetitive region from E. australis MaSp sequence (GNSGRGQGGYGQGSGGNAAAAAAAAAAAAAAAGQGGQGGYGRQSQGAGSAAAAA AAAAAAAAAAAAGSGQGGYGGQGQGGYGQSGNS) or (GNSGRGQGGYGQGSGGNAAAAAAAAAAAAAAAGQGGQGGYGRQSQGAGSAAAAA AAAAAAAAAAAPGNSGRGQGGYGQGSGGNAAAAAAAAAAAAAAAGQGGQGGYGRQ SQGAGSAAAAAAAAAAAAAAAAAGSGQGGYGGQGQGGYGQSGNS) or (GNSGRGQGGYGQGSGGNAAAAAAAAAAAAAAAGQGGQGGYGRQSQGAGSAAAAA AAAAAAAAAAAPGNSGRGQGGYGQGSGGNAAAAAAAAAAAAAAAGQGGQGGYGRQ SQGAGSAAAAAAAAAAAAAAAAPGNSGRGQGGYGQGSGGNAAAAAAAAAAAAAAA GQGGQGGYGRQSQGAGSAAAAAAAAAAAAAAAAAGSGQGGYGGQGQGGYGQSGNS) or C. clavipes F1Sp sequence (DTSGPGQYYRSSSSGGGGGGQGGPVVTEGPGGAGPGGYGPGGSGPGGYGPGGSGPGG YGPGGSGPGGYGPGGSGPGGYGPGGSGPGGYGPGGYGPGGSGPGGYGPGGTGPGGSGP GGYGPGGSGPGGSGPGGYGPGGSGPGGFGPGGSGPGGYGPGGSGPGGAGPGGVGPGGF GPGGAGPGGAGPGGAGPGGAGPGGAGPGGAGPGGAGPGGAGPGGAGPGGAGPGGAG GAGGAGGAGGSGGAGGSGGTTIIEDLDITIDGADGPITISEELTISGAGGSGPGGAGPGGV GPGGSGPGGVGPGGSGPGGVGPGGSGPGGVGPGGAGGPYGPGGSGPGGAGGAGGPGG AYGPGGSYGPGGSGGPGGAGGPYGPGGEGPGGAGGPYGPGGAGGPYGPGGAGGPYGP GGEGGPYGPGGSYGPGGAGGPYGPGGPYGPGGEGPGGAGGPYGPGGVGPGGSGPGGA AA) or from TuSp sequence (SAARSGAQSSSTTTTSSTSGSQAASSSASQASASSFAQASSASLAASSSESSAFSSANTLSA LGNVAYQLGFNVANTLGLGNAAGLGAALSQAVSSVGVGASSGTYANAVSNAVGQFLA GQGILNGANAASLASSFASALSASAASVASSSAAQSASQSQAAASAFSRAASQSASQAA A), and a C-terminal cysteine (C) or cysteninealanine (CysAla). The underlined are additional amino acids stemming from the Not1 restriction site.

The recombinant spidroin constructs were cloned into pET28a (+) plasmids. The plasmids were used to transform BL21 (DE) 3 E. coli competent cells by heat shock, followed by overnight incubation on Luria broth (LB) plates containing 50 μg/mL kanamycin (kan). From there, a single colony was inoculated in 50 mL of LB medium containing 50 μg/mL kan and grown overnight at 25° C. The overnight culture was afterwards diluted in 1 L LB culture with antibiotic to OD₆₀₀ 0.01 and poured in baffled 2.5 L flasks, which were incubated at 37° C. Upon reaching OD₆₀₀ 0.6 the temperature was reduced to 25° C. and the expression was induced using 0.05 mM isopropyl β-D-1-thiogalactopyranoside (IPTG). After overnight expression (F4M and F6M constructs were expressed only for 4 h), the cells were harvested by centrifugation at 7000×g for 15 min and stored at −20° C. until further use.

The cell pellets were resuspended in 10 mL of immobilized metal affinity chromatography (IMAC) loading buffer (20 mM sodium phosphate, pH 7.2, 300 mM NaCl, 15 mM imidazole) per gram and lysed by ultrasonication. Soluble cell lysate fraction was separated by centrifugation for 40 min at 30,000×g, 4° C. and filtered through a 0.22 μm pore-sized filter. The lysate was loaded on HisTrap HP column (Cytivia) and eluted using 20 mM sodium phosphate, pH 7.2, 300 mM NaCl, 350 mM imidazole. The purity was further improved by gel filtration using a 16/600 Superdex PG200 size exclusion column in 20 mM sodium phosphate, pH 7.2, 300 mM NaCl.

### General Synthesis

Recombinant spidroins bioconjugates 1.1-1.15 were prepared according to Scheme 1. Recombinant spidroin was reduced and reacted with PEG-Mal to produce the products.

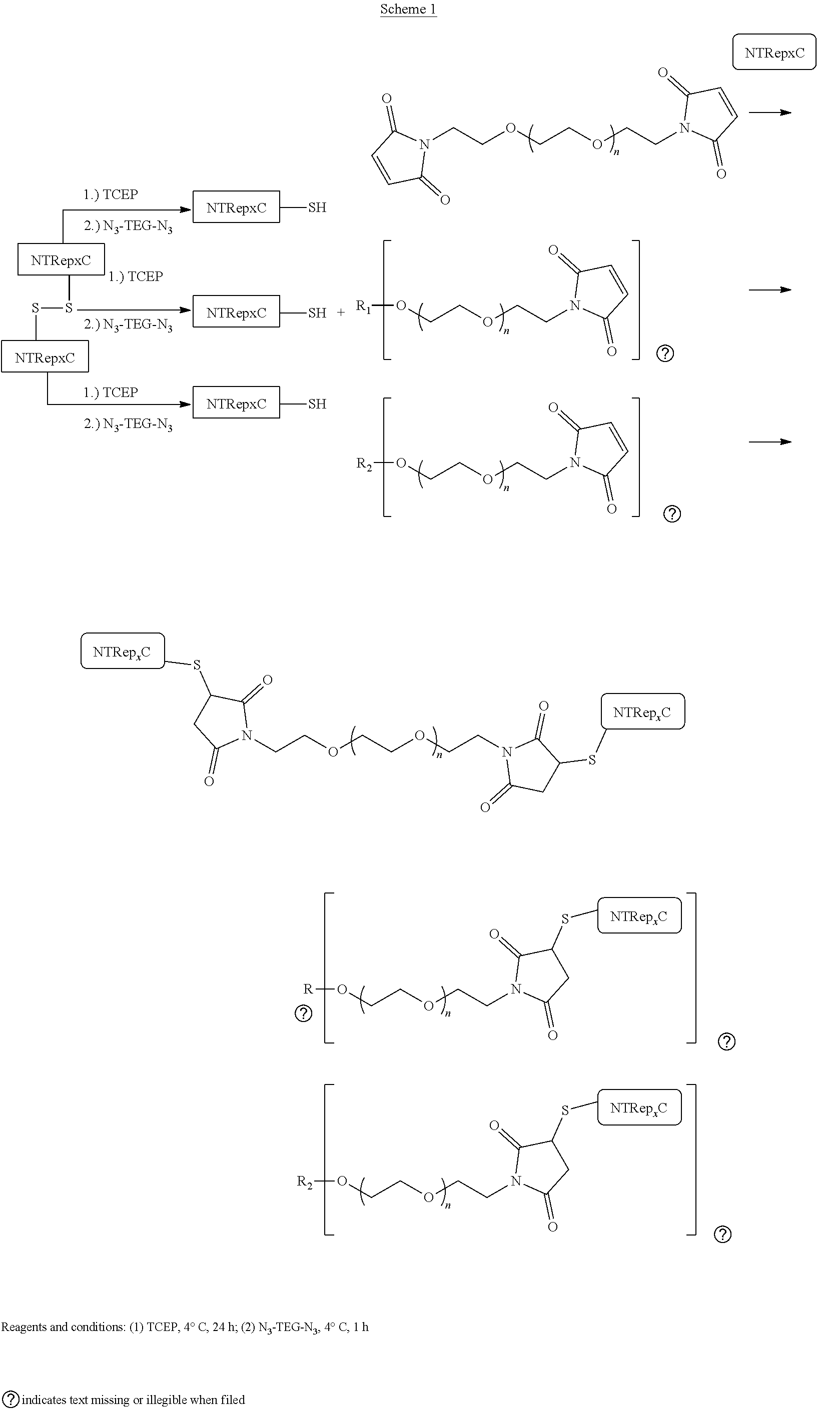

Synthesis of products 1.1-1.15, general method. Exemplified by the synthesis of FIF-2-8arm 1.9-1.11

To a solution of FIF protein (0.6 mM solution, 1 mL, 0.0006 mmol, 1 eq) in phosphate buffer (c=20 mM, pH=7.2) were added TCEP solution (50 mM, 0.12 mL, 0.006 mmol, 10 eq) in phophate buffer (c=20 mM, pH=7.2). The mixture was quickly vortexed and left at 4° C. for 24 hours. After that 1,2-bis(2-azidoethoxy) ethane (6 mg, 0.030 mmol, 5 eq) were added and mixture was kept for an additional hour. Then the corresponding PEG-Mal solution in phosphate buffer (c=20 mM, pH=7.2) were added and after ˜1 h the mixture was analyzed by SDS-PAGE.

Recombinant spidroin bioconjugation with PEG (Maleimide) 2 with MW 5,000 Da. Reduced protein was treated with PEG (Maleimide) 2 with MW 5000 Da (1.5 mg, 0.0003 mmol, 0.43 eq) solution in phosphate buffer (c=20 mM, pH=7.2, 0.130 mL). Relative ratios: bis-conjugated PEG 44%, mono-conjugated PEG 39%, unreacted Flag 12%.

Recombinant spidroin bioconjugation with PEG (Maleimide) 4 with MW 10000 Da. Reduced protein was treated with PEG (Maleimide) 4 with MW 10000 Da (1.5 mg, 0.0002 mmol, 0.22 eq) solution in phosphate buffer (c=20 mM, pH=7.2, 0.130 mL). Relative ratios: tetra-conjugated PEG 13%, tri-conjugated PEG 18%, bis-conjugated PEG 19%, mono-conjugated PEG 12%, unreacted Flag 38%.

Recombinant spidroin bioconjugation with PEG (Maleimide) 8 with MW 10000 Da. Reduced protein was treated with PEG (Maleimide) 8 with MW 10000 Da (0.75 mg, 0.00008 mmol, 0.11 eq) solution in phosphate buffer (c=20 mM, pH-7.2, 0.065 mL). Relative ratios: poli-conjugated PEG (5-8 proteins per PEG) 33%, tetra-conjugated PEG 13%, tri-conjugated PEG 13%, bis-conjugated PEG 10%, unreacted FIF 31%.

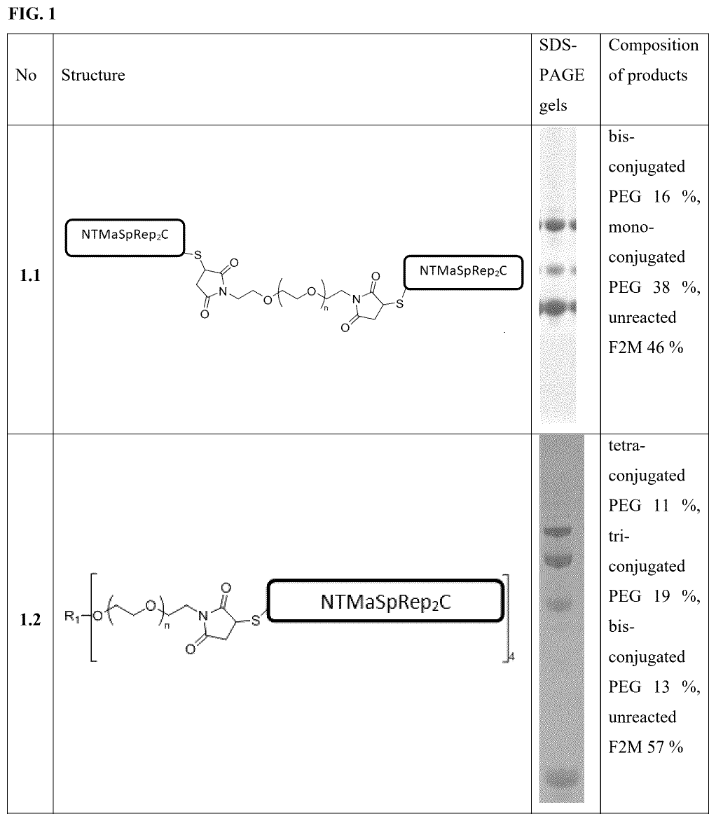

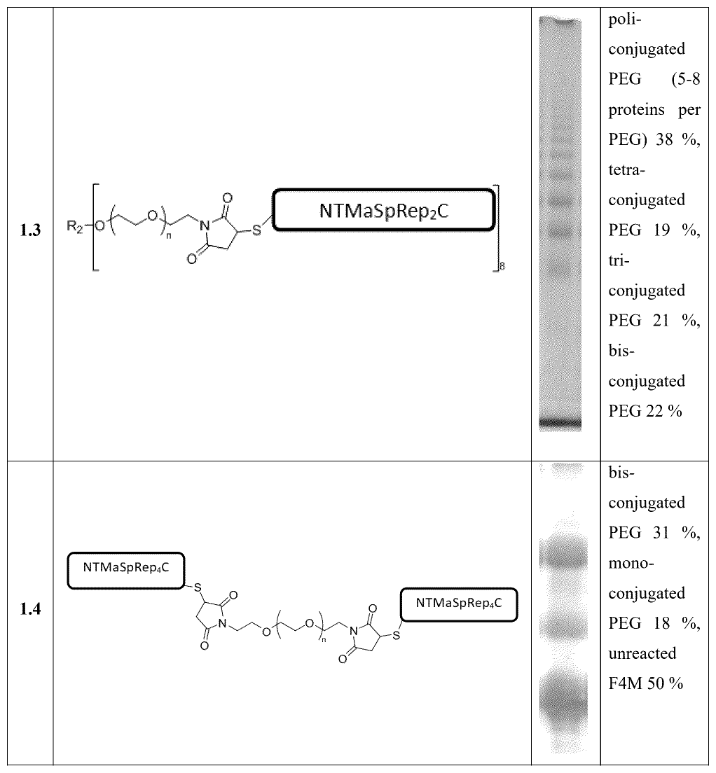

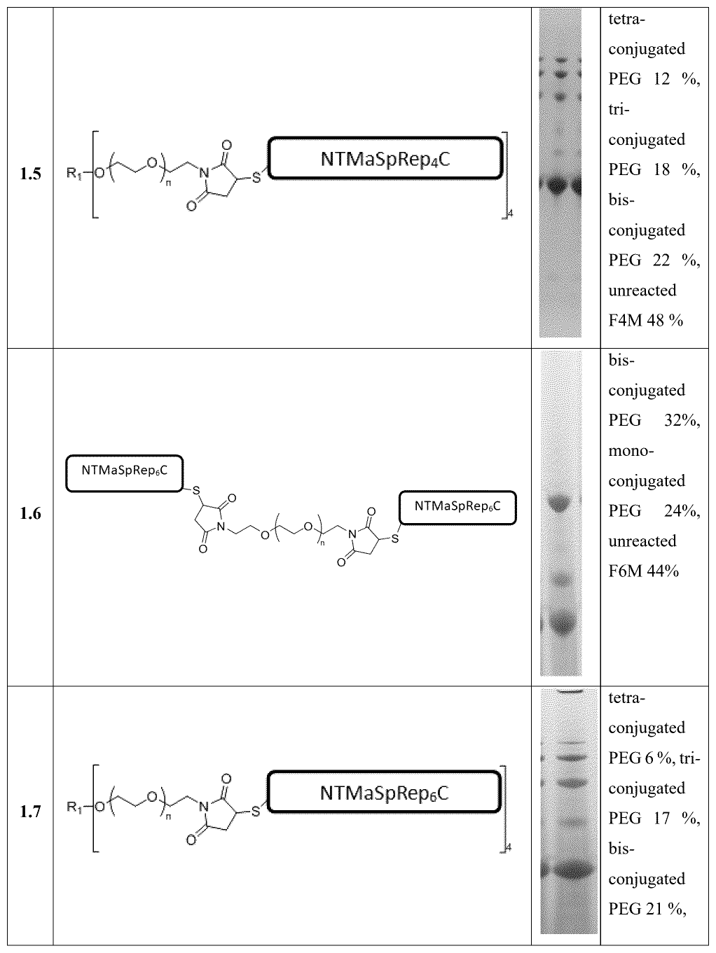

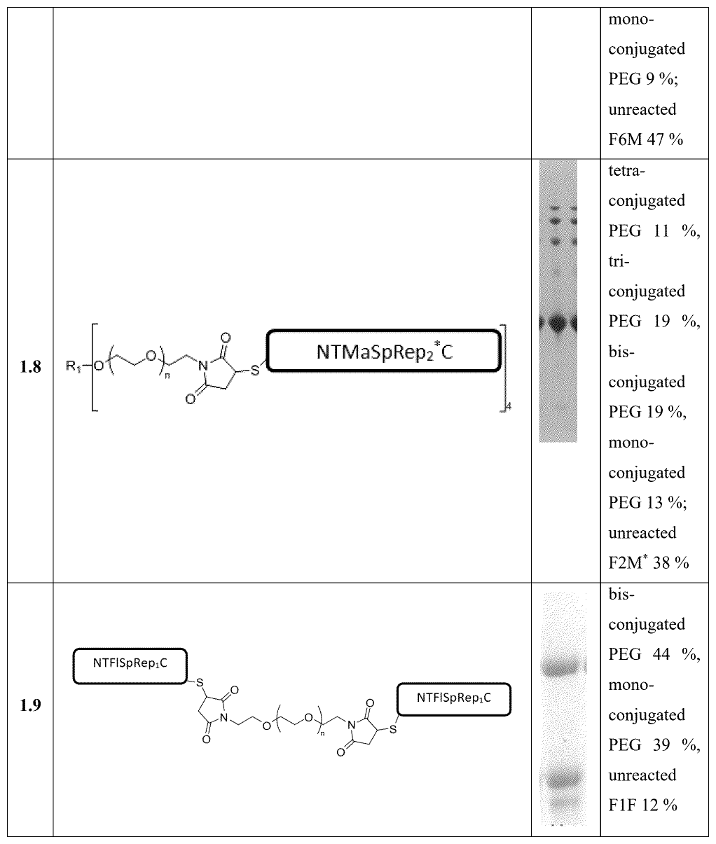

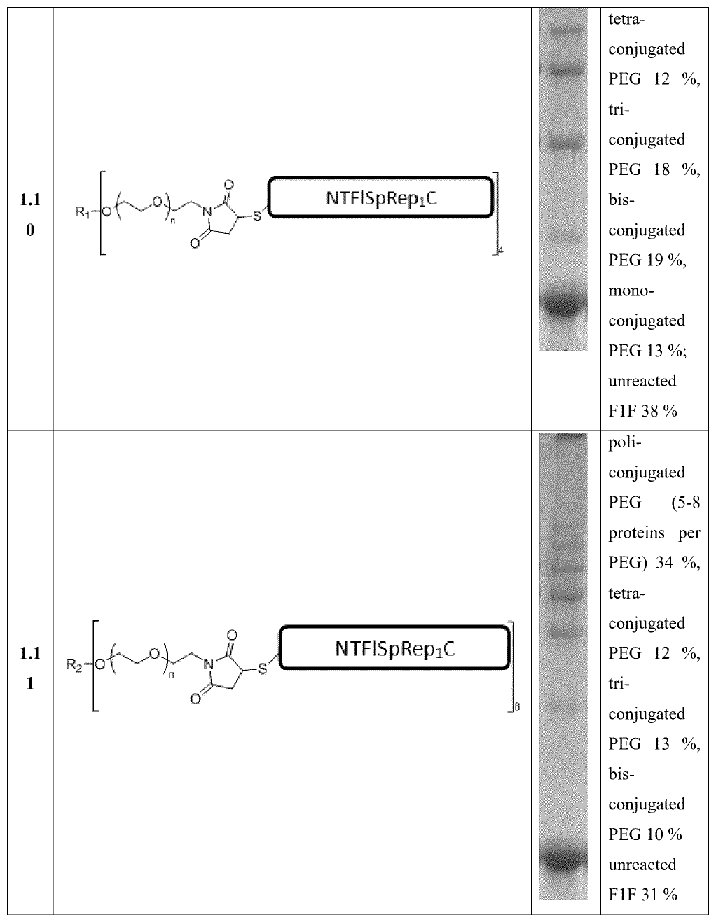

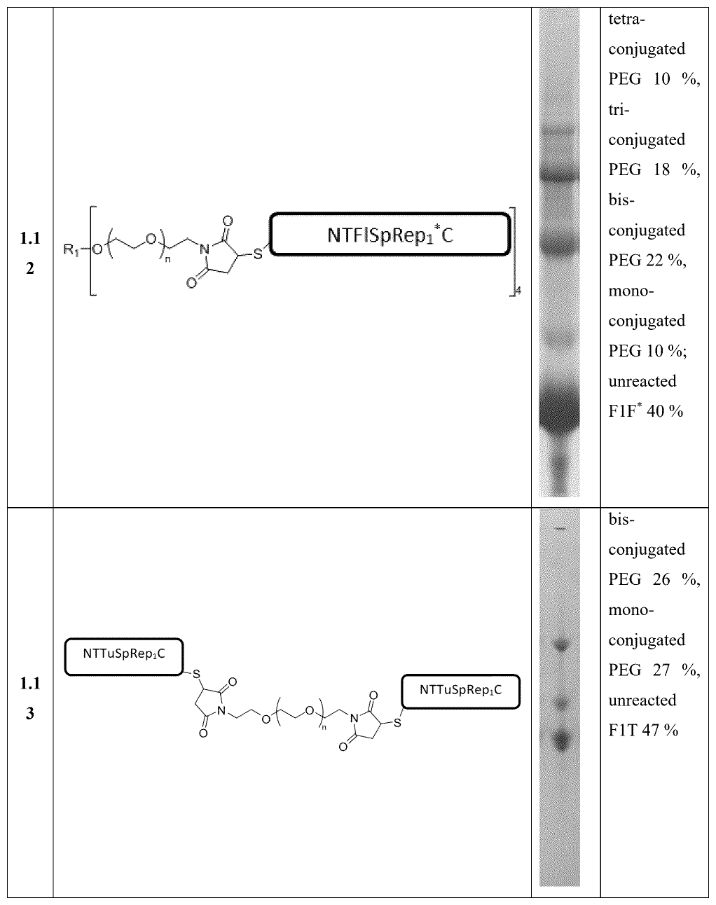

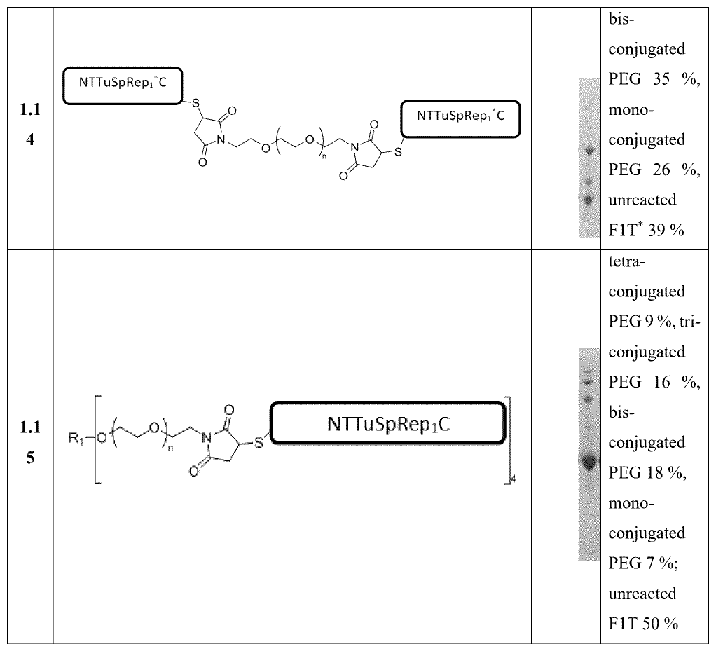

By the method described above the compounds as described in FIG. 1 were obtained.

### Fiber Spinning

The FIF spidroin was dialyzed against pH 7.2, 20 mM sodium phosphate buffer and concentrated to 300 mg/mL. The dope was then injected into a coagulation buffer (pH 5, 500 mM sodium acetate, 200 mM NaCl) using a syringe pump and collected on a spinning frame as described in Andersson et al. Nat. Chem. Biol. 2017, 13, 262-264.

1. A recombinant spidroin comprising from 244 to 623 amino acid residues and defined by the formula NT-(Rep)ₓ-C wherein:
an N-terminal domain NT consisting of 130 to156 amino acid residues derived from the N-terminal domain of a spidroin;
a repetitive domain (Rep)ₓ consisting of from 87 to 463 amino acid residues derived from the repetitive sequences in a spidroin, wherein x is a number of repetitive sequences from 1 to 8;
a domain C is Cys (Z) residue, where Z is optional, if present, is selected from the group Ala, Arg, Asn, Asp, Cys, Glu, Gln, Gly, His, Ile, Leu, Lys, Met, Phe, Pro, Ser, The, Trp, Tyr, Val.

2. The recombinant spidroin according to claim 1, wherein a domain C is Cys.

3. The recombinant spidroin according to claim 1, wherein a domain Cis CysAla.

4. The recombinant spidroin according to claim 1, wherein (Rep)ₓ-domain preferably consists of 174-463 amino acid residues.

5. A method of bioconjugation of the recombinant spidroin comprising the steps of:
a) reducing the disulfide bond to obtain free thiol group with tris(2-carboxyethyl) phosphine, wherein the reaction temperature is in range from 2 to 10° C. and pH is in the range from 6.0 to 9.0.
b) quenching of obtained product in step a) with 1,2-bis(2-azidoethoxy)ethane;
c) coupling the product obtained in step b) with 2- to 8-arm polyethylene glycol maleimide (PEG-Mal);
d) forming the bioconjugated recombinant spidroin.

6. The method according to claim 5 wherein the pH in step a) is preferably in range from 7.2 to 7.5.

7. The method according to claim 5 wherein the temperature in step a) is preferably in range from 3° C. to 5° C.

8. A bioconjugated recombinant spidroin comprising from 244 to 623 amino acid residues and defined by the formula (NT-(Rep)ₓ-C)_{y}-PEG-Mal wherein:
an N-terminal domain NT consisting of 130 to 156 amino acid residues derived from the N-terminal domain of a spidroin;
a repetitive domain (Rep) consisting of 87 to 463 amino acid residues derived from the repetitive sequences in a spidroin, wherein x is a number of repetitive sequences from 1 to 8;
a domain C is Cys (Z) residue, where Z is optional, if present, is selected from the group Ala, Arg, Asn, Asp, Cys, Glu, Gln, Gly, His, Ile, Leu, Lys, Met, Phe, Pro, Ser, The, Trp, Tyr, Val;
y is a number of bioconjugated NT-(Rep)ₓ-C proteins from 1 to 8;
PEG-Mal is 2- to 8-arm polyethylene glycol maleimide;
and optical isomers thereof.

9. The bioconjugated recombinant spidroin according to claim 8 which has a structure: 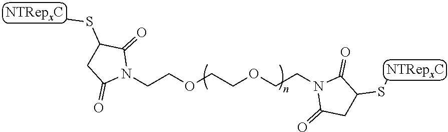
wherein:
n is an integer selected from 50 to 400, corresponding to an average molecular weight between 5000 Da and 10000 Da;
x is a number of repetitive sequences from 1 to 8;
C is Cys (Z) residue where Z is optional, if present, is selected from the group Ala, Arg, Asn, Asp, Cys, Glu, Gln, Gly, His, Ile, Leu, Lys, Met, Phe, Pro, Ser, The, Trp, Tyr, Val.

10. The bioconjugated recombinant spidroin according to claim 8 which has a structure: 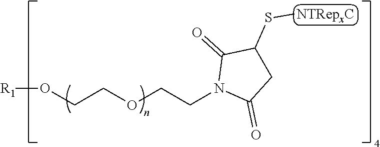
wherein:
R1 is pentaerythritol core;
n is an integer selected from 50 to 400, corresponding to an average molecular weight between 5000 Da and 10000 Da;
x is a number of repetitive sequences from 1 to 8;
C is Cys (Z) residue where Z is optional, if present, is selected from the group Ala, Arg, Asn, Asp, Cys, Glu, Gln, Gly, His, Ile, Leu, Lys, Met, Phe, Pro, Ser, The, Trp, Tyr, Val.

11. The bioconjugated recombinant spidroin according to claim 8 which has a structure: 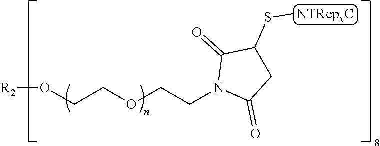
R2 is tripentaerythritol core;
n is an integer selected from 50 to 400, corresponding to an average molecular weight between 5000 Da and 10000 Da;
x is a number of repetitive sequences from 1 to 8;
C is Cys (Z) residue where Z is optional, if present, is selected from the group Ala, Arg, Asn, Asp, Cys, Glu, Gln, Gly, His, Ile, Leu, Lys, Met, Phe, Pro, Ser, The, Trp, Tyr, Val.

12. A bioconjugated recombinant spidroins for use in regenerative medicine.

13. The bioconjugated recombinant spidroins according to claim 12 for use as a cell culture scaffold.


SEQUENCE LISTING (8 sequences)

SEQ ID NO: 1 - protein, 244 aa, Artificial Sequence
  F2M
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVGNSGRGQGGYGQGSGGNAAAAAAAAAAAAAAAGQGGQGGYGRQSQGAGSAAAAAAAAAAAAAAAAAGSGQGGYGGQGQGGYGQSGNSC

SEQ ID NO: 2 - protein, 310 aa, Artificial Sequence
  F4M
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVGNSGRGQGGYGQGSGGNAAAAAAAAAAAAAAAGQGGQGGYGRQSQGAGSAAAAAAAAAAAAAAAAPGNSGRGQGGYGQGSGGNAAAAAAAAAAAAAAAGQGGQGGYGRQSQGAGSAAAAAAAAAAAAAAAAAGSGQGGYGGQGQGGYGQSGNSC

SEQ ID NO: 3 - protein, 376 aa, Artificial Sequence
  F6M
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVGNSGRGQGGYGQGSGGNAAAAAAAAAAAAAAAGQGGQGGYGRQSQGAGSAAAAAAAAAAAAAAAAPGNSGRGQGGYGQGSGGNAAAAAAAAAAAAAAAGQGGQGGYGRQSQGAGSAAAAAAAAAAAAAAAAPGNSGRGQGGYGQGSGGNAAAAAAAAAAAAAAAGQGGQGGYGRQSQGAGSAAAAAAAAAAAAAAAAAGSGQGGYGGQGQGGYGQSGNSC

SEQ ID NO: 4 - protein, 245 aa, Artificial Sequence
  F2M
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVGNSGRGQGGYGQGSGGNAAAAAAAAAAAAAAAGQGGQGGYGRQSQGAGSAAAAAAAAAAAAAAAAAGSGQGGYGGQGQGGYGQSGNSCA

SEQ ID NO: 5 - protein, 620 aa, Artificial Sequence
  F1F
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVDTSGPGQYYRSSSSGGGGGGQGGPVVTEGPGGAGPGGYGPGGSGPGGYGPGGSGPGGYGPGGSGPGGYGPGGSGPGGYGPGGSGPGGYGPGGYGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAGPGGAGPGGAGPGGAGPGGAGPGGAGPGGAGPGGAGPGGAGPGGAGGAGGAGGAGGSGGAGGSGGTTIIEDLDITIDGADGPITISEELTISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGSGPGGAAAC

SEQ ID NO: 6 - protein, 621 aa, Artificial Sequence
  F1F
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVDTSGPGQYYRSSSSGGGGGGQGGPVVTEGPGGAGPGGYGPGGSGPGGYGPGGSGPGGYGPGGSGPGGYGPGGSGPGGYGPGGSGPGGYGPGGYGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAGPGGAGPGGAGPGGAGPGGAGPGGAGPGGAGPGGAGPGGAGPGGAGGAGGAGGAGGSGGAGGSGGTTIIEDLDITIDGADGPITISEELTISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGSGPGGAAACA

SEQ ID NO: 7 - protein, 336 aa, Artificial Sequence
  F1T
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVSAARSGAQSSSTTTTSSTSGSQAASSSASQASASSFAQASSASLAASSSFSSAFSSANTLSALGNVAYQLGFNVANTLGLGNAAGLGAALSQAVSSVGVGASSGTYANAVSNAVGQFLAGQGILNGANAASLASSFASALSASAASVASSSAAQSASQSQAAASAFSRAASQSASQAAAC

SEQ ID NO: 8 - protein, 337 aa, Artificial Sequence
  F1T
MGKHHHHHHPMSDYDIPTTENLYFQGIANSPFSNPNTAEAFARSFVSNIVSSGEFGAQGAEDFEDIIQSLIQAQSMGKGRHDTKAKAKAMQVALASSIAELVIAESSGGDVQRKTNVISNALRNALMSTTGSPNEEFVHEVQDLIQMLSQEQINEVSAARSGAQSSSTTTTSSTSGSQAASSSASQASASSFAQASSASLAASSSFSSAFSSANTLSALGNVAYQLGFNVANTLGLGNAAGLGAALSQAVSSVGVGASSGTYANAVSNAVGQFLAGQGILNGANAASLASSFASALSASAASVASSSAAQSASQSQAAASAFSRAASQSASQAAACA